(19) United States
(12) Patent Application Publication
(10) Pub. No.: US 2025/0339099 A1
(43) Pub. Date: Nov. 6, 2025

(54) ADHERABLE ANCHOR FOR WEARABLE MEDICAL SENSOR

(71) Applicant: Becton, Dickinson and Company, Franklin Lakes, NJ (US)

(72) Inventors: Julio Agustin Isla Garcia, Irvine, CA (US); Andres S. Aguirre, Mission Viejo, CA (US); Timothy John Payne, Santa Ana, CA (US); Blake W. Axelrod, Irvine, CA (US)

(21) Appl. No.: 19/265,851

(22) Filed: Jul. 10, 2025

Related U.S. Application Data
(63) Continuation of application No. PCT/US2024/011061, filed on Jan. 10, 2024.
(60) Provisional application No. 63/479,261, filed on Jan. 10, 2023.

(51) Int. Cl. A61B 5/00

(52) U.S. Cl. CPC A61B 5/68335; A61B 5/6835; A61B 5/6844

(57) ABSTRACT

A fastening device is disclosed for attaching a wearable sensor to a body of a patient, the wearable sensor comprising a sensor configured to measure characteristics inside the patient's body. The fastening device includes an adherable anchor configured to affix the wearable sensor to the body of the patient via an adhesive surface and a handle configured to expose the adhesive surface when pulled following attachment of the adherable anchor to the wearable medical sensor. The adherable anchor includes an attachment portion configured to secure the adherable anchor to the wearable medical sensor and an adhesive surface configured to affix the adherable anchor to the body of the patient. The handle is attached to the adhesive surface.

### CROSS-REFERENCE TO RELATED APPLICATION(S)

This application is a continuation of International Application No. PCT/US2024/011061, filed Jan. 10, 2024, entitled “ADHERABLE ANCHOR FOR WEARABLE MEDICAL SENSOR,” which claims the benefit of U.S. Provisional Application No. 63/479,261, filed Jan. 10, 2023, and entitled “ADHERABLE ANCHOR FOR WEARABLE MEDICAL SENSOR,” the disclosures of which are hereby incorporated by reference in their entireties.

### BACKGROUND

The present disclosure is generally directed to wearable medical sensors and more particularly to adhering wearable medical sensors to a patient for long-term monitoring.

A variety of wearable medical sensors are used in hospital and outpatient settings for long-term continuous or intermittent motoring. Wearable medical sensors do not require operator assistance to maintain contact between the wearable medical sensor and skin of the patient, but often require an adhesive mechanism to securely affix the wearable medical sensor to the skin. Adhesive patches or bandages can be applied over wearable medical sensors with some success; however, they can be difficult to properly apply. Many sensors need to be moved on the patient to identify the target position for monitoring before being adhered in place. Sensors that include adhesive features can require lifting the sensor from the patient's body to expose the adhesive, which risks losing track of the target position and can increase the time to deployment. It can be particularly important to reduce the risk of improper placement and time required for deployment in preparation for surgery.

### SUMMARY

A fastening device is disclosed for attaching a wearable sensor to a body of a patient, the wearable sensor comprising a sensor configured to measure characteristics inside the patient's body. The fastening device includes an adherable anchor configured to affix the wearable sensor to the body of the patient via an adhesive surface and a handle configured to expose the adhesive surface when pulled following attachment of the adherable anchor to the wearable medical sensor. The adherable anchor includes an attachment portion configured to secure the adherable anchor to the wearable medical sensor and an adhesive surface configured to affix the adherable anchor to the body of the patient. The handle is attached to the adhesive surface.

A method of attaching a wearable sensor to a body of a patient is disclosed, the wearable sensor comprising a sensor configured to measure characteristics inside the patient's body. The method includes attaching a fastening device to the wearable sensor. The fastening device includes an adherable anchor configured to affix the wearable sensor to skin of the body of the patient and a handle comprising a release liner attached to the adherable anchor. The method further includes positioning the wearable sensor on the body of the patient and pulling a handle of the fastening device to remove the release liner from an adhesive surface of the adherable anchor.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a perspective bottom view of fastening device for anchoring a wearable sensor to a body of a patient.

FIG. 2 is perspective side view of the fastening device of FIG. 1.

FIG. 3 is perspective top view of the fastening device of FIG. 1.

FIG. 4 is a perspective side view of the fastening device of FIGS. 1-3 coupled to a wearable medical sensor.

FIG. 5 is a perspective top view of an adherable anchor of the fastening device coupled to the wearable sensor following deployment.

FIGS. 6A-6C are perspective views of steps of a method for affixing the wearable sensor of FIG. 3 to a body of a patient with the fastening device.

FIG. 7A is a schematic diagram of a wearable medical sensor attached to a body of a patient by a fastening device.

FIG. 7B is a schematic diagram of a wearable medical sensor attached to a body of a patient by a fastening device and angled toward a target area of interest.

FIG. 8A is a schematic diagram of a wearable medical sensor attached to a body of patient by an adherable anchor having an inflatable bladder in a deflated position.

FIG. 8B is a schematic diagram of a wearable medical sensor attached to a body of patient by the adherable anchor of FIG. 8A with the inflatable bladder in an inflated position.

FIG. 8C is a top view of the inflatable bladder of FIGS. 8A and 8B positioned on a wearable medical sensor.

FIG. 8D is a top view of an alternative inflatable bladder positioned on the wearable medical sensor of FIGS. 8A and 8B for application with the adherable anchor.

FIG. 8E is top view of yet another alternative inflatable bladder positioned on the wearable medical sensor of FIGS. 8A and 8B for application with the adherable anchor.

FIG. 9A is a schematic diagram of a wearable medical sensor attached to a body of a patient by an adjustable adherable anchor having one or more threaded fasteners for tilting a sensor face of the wearable medical sensor toward a target area of interest.

FIG. 9B is a schematic diagram of a wearable medical sensor attached to a body of a patient by the adherable anchor of FIG. 9A with the one or more threaded fasteners adjusted to tilt the sensor face toward a target area of interest.

FIG. 9C is a top view of the adherable anchor of FIGS. 9A and 9B applied to a wearable medical sensor.

FIG. 10 is a schematic diagram of another embodiment of a wearable medical sensor attached to a body of a patient by an adherable anchor.

FIG. 11A is a schematic diagram of the wearable medical sensor and adherable anchor of FIG. 10 having a wedge-shaped block disposed therebetween to cause a sensor face of the wearable medical sensor to tilt at a first tilt angle.

FIG. 11B is a side view of the wedge-shaped block of FIG. 11A.

FIG. 11C is a top view of the wedge-shaped block of FIG. 11A.

FIG. 12A is a schematic diagram of the wearable medical sensor and adherable anchor of FIG. 10 having a wedge-shaped block disposed therebetween to cause a sensor face of the wearable medical sensor to tilt at a second tilt angle.

FIG. 12B is a side view of the wedge-shaped block of FIG. 12A.

FIG. 12C is a top view of the wedge-shaped block of FIG. 12A.

FIG. 13A is a schematic diagram of another embodiment of an adherable anchor applied to a wearable medical sensor and a body of a patient.

FIG. 13B is a schematic top view of the adherable anchor of FIG. 13A.

While the above-identified figures set forth embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps and/or components not specifically shown in the drawings.

### DETAILED DESCRIPTION

The present disclosure is directed to a fastening device for affixing a wearable medical sensor to a patient's body for long-term monitoring. As used herein, long-term monitoring refers to monitoring for a period of time greater than a few minutes and which does not require operator assistance to maintain contact between the wearable sensor and skin of the patient. Wearable sensors can be anchored to the skin for continuous or intermittent monitoring at a target location at which they are located.

Wearable medical sensors can include but are not limited to ultrasonic sensors used, for example, for measuring vessel pulsatility, blood flow within a vessel or vessel network, and tracking of other biological features such as organ motion or respiration rate; phototplethysmogram (PPG) sensors for monitoring pulse rate (PR), heart rate (HR), variability, pulse oximetry (SpO₂), cardiac output (C), and blood pressure (BP); near infrared spectroscopy (NIRS) sensors for monitoring hemoglobin (Hb), hematocrit (HCT), and tissue oximetry (StO₂); temperature sensors; electrocardiogram (ECG) sensors; electroencephalogram (EEG) sensors; acoustic sensors; ballistocardiogram (BCG) sensors; minimally invasive sensors such as glucose monitors (CGM) and lactate monitors; and combinations thereof. For example, wearable medical sensors according to the present disclosure can include multiple sensors in a single patch (e.g., PPG, ECG, and temperature, or sensors monitoring a plurality of vital signs). The present disclosure shows a fastening device configured for use with a wearable ultrasound transducer. Information obtained by the ultrasound transducer can be utilized to infer and/or predict possible states of the human and/or animal body, like hypertension and hemorrhage, as well as other physiological variables, like stroke volume, stroke volume variations, and cardiac output. It will be understood by one of ordinary skill in the art that the disclosed fastening device can be configured for use with a wide variety of wearable medical sensors including but not limited to those listed above.

The fastening device of the present disclosure is configured for use with wearable medical sensors that are relatively thin and flat in profile, and which can be secured to a patient's body by an adherable anchor disposed over top of the wearable medical sensor and extending to skin surrounding the wearable medical sensor.

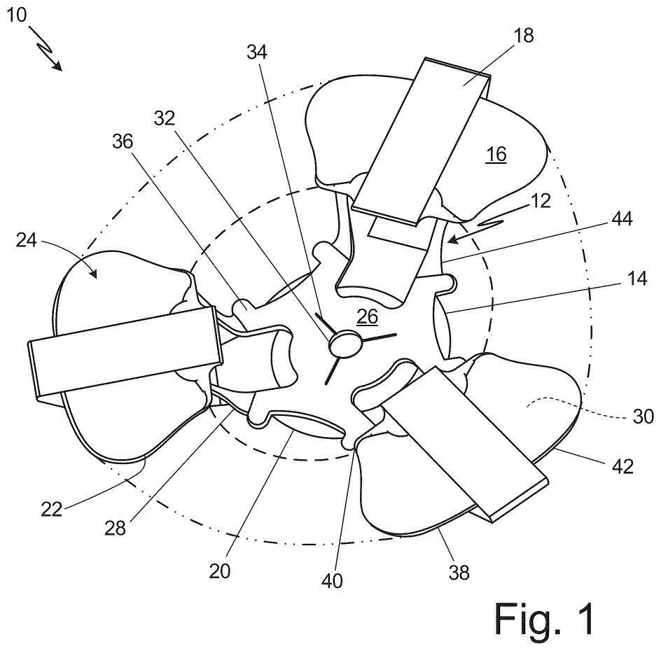

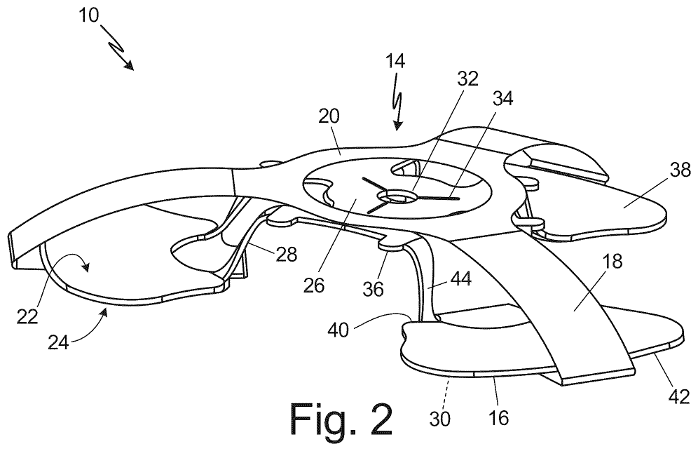

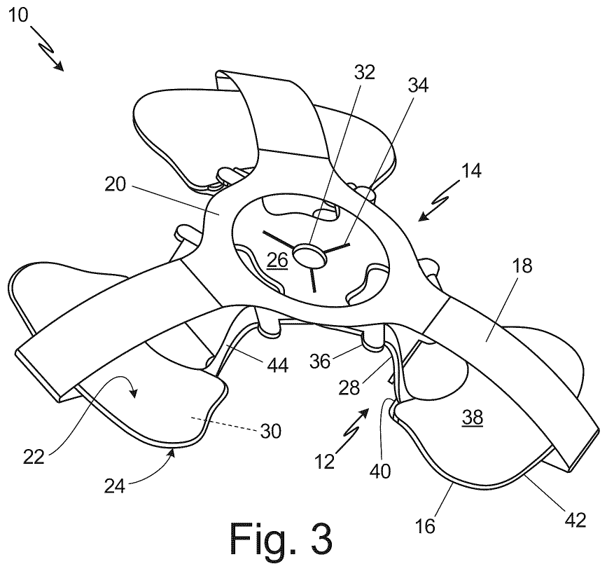

FIG. 1 is a perspective bottom view of fastening device 10 for anchoring a wearable medical sensor to a body of a patient. FIG. 2 is a perspective side view of fastening device 10. FIG. 3 is perspective top view of fastening device 10. FIGS. 1-3 are discussed together herein. Fastening device 10, adherable anchor 12, handle 14, release liners 16, release strips 18, junction 20, top side 22, bottom side 24, attachment portion 26, arms 28, adhesive layer 30, hole 32, slits 34, anti-rotation member 36, end bodies 38, inner edge 40, outer edge 42, and struts 44 are shown.

Handle 14 is attached to adherable anchor 12 via release liners 16 and is configured for use in deploying adherable anchor 12. Handle 14 includes release strips 18 connected at junction 20. Release strips 18 are connected to release liners 16. Adherable anchor 12 has top side 22 and bottom side 24. Handle 14 extends over top side 22 of adherable anchor 12 and is spaced therefrom. Adherable anchor 12 includes attachment portion 26, arms 28, and adhesive layer 30. Attachment portion 26 can include hole 32 and slits 34. Adhesive layer 30 is disposed on bottom side 24 of arms 28. Release liners 16 are disposed on adhesive layer 30. Adhesive layer 30 can be disposed on end bodies 38 of arms 28. End bodies 38 can include inner edge 40 and outer edge 42. Arms 28 can include struts 44.

Adherable anchor 12 is configured for deployment over a top surface of a wearable medical sensor such that adherable anchor 12 does not interfere with a sensor face positioned against the body of the patient or functioning of the wearable medical sensor. Adhesive layer 30 is configured to affix adherable anchor 12 and thereby the wearable medical sensor to a patient's body. Adherable anchor 12 can be a unitary body formed of a material having a flexibility sufficient to allow movement of arms 28 relative to attachment portion 26 while having a rigidity sufficient to maintain a shape and position of arms 28 relative to attachment portion 26 when forces are removed. As such, a practitioner does not need to position arms 28 for deployment. Arms 28 are automatically positioned for deployment when fastening device 10 is attached to the wearable medical sensor.

Fastening device 10 can be used to affix a wearable medical sensor to a patient's body with case. As further described herein, adherable anchor 12 can be secured to the patient's body by simply pulling handle 14 once the wearable medical sensor with attached fastening device 10 is in a position of placement. Unlike prior art adhesive patches or bandages, fastening device 10 does not require a practitioner to remove multiple release liners independently or to adjust, position, or stretch adhesive patches or bandages to secure the wearable medical sensor with an optimal applied force required to maintain a connection between a sensor face and the patient's body.

Adherable anchor 12 includes attachment portion 26. Attachment portion 26 is configured to secure adherable anchor 12 to a wearable medical sensor. In some examples, attachment portion 26 can include hole 32 and a plurality of slits 34 configured to receive a mounting member protruding from a top surface of the wearable medical sensor. Slits 34 are cuts in adherable anchor 12 configured to temporarily expand a size of hole 32 for attachment and/or detachment of the wearable medical sensor. Slits 34 extend outward from hole 32. Slits 34 can be uniformly spaced about hole 32. The amount and lengths of slits 34 can be selected to provide suitable retention of adherable anchor 12 on the wearable medical sensor and to allow a practitioner to attach and detach adherable anchor 12 from the wearable medical sensor without significant difficulty. In some examples, the wearable medical sensor can include a raised nub having a diameter or perimeter greater than a diameter or perimeter of hole 32, as discussed further herein. Attachment portion is not limited to the combination of hole 32 and slits 34. Other mechanical attachment features are contemplated. Any attachment feature should be capable of being securely fastened to the wearable medical sensor and the attachment should not be interrupted or compromised by movement of the patient.

Alternatively, or in addition to mechanical fastening features, attachment portion 26 can include an adhesive layer. To limit damage to the wearable medical sensor, the adhesive layer can be, for example, a removable pressure sensitive adhesive (PSA).

Attachment portion 26 can have a planar surface configured to interface with a top surface of the wearable medical sensor. Attachment portion 26 can have any suitable shape. As discussed further herein, attachment portion 26 can be designed to substantially cover a top surface of the wearable medical sensor such that attachment portion 26 exerts a downward force across a surface of the wearable medical sensor when adherable anchor 12 is affixed to the patient's body. As shown in FIG. 1, attachment portion has a substantially round shape, however, other shapes are contemplated and can vary depending on the shape of the wearable medical sensor.

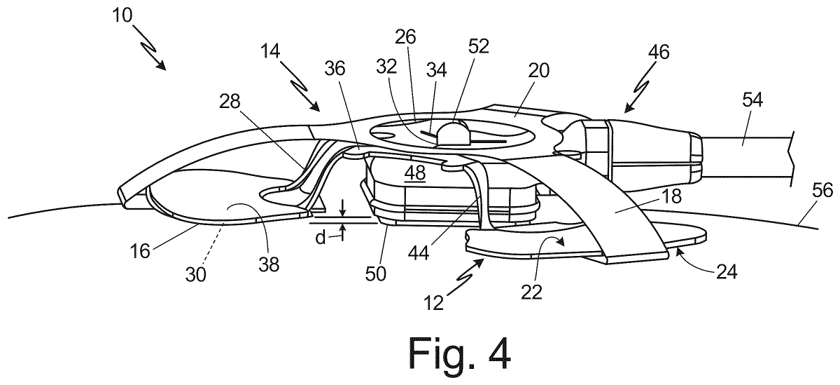

Adherable anchor 12 can include one or more anti-rotation members 36 configured to prevent rotation of adherable anchor 12 with respect to the wearable medical sensor after fastening device 10 has been secured to the wearable medical sensor and to prevent rotation of the wearable medical sensor once adherable anchor 12 has been attached to the body of the patient. Anti-rotation members 36 can include, for example, tabs that extend outward from attachment portion 26 of adherable anchor 12 on opposite sides of a feature (e.g., cable junction shown in FIG. 4) of the wearable medical sensor to restrict movement of the feature relative to adherable anchor 12 and vice versa and retain a position of the wearable sensor with respect to adherable anchor 12.

A plurality of arms 28 extends outward from attachment portion 26. Typically, at least three arms 28 can be included to provide two-dimensional axial control and prevent sliding of the wearable medical sensor once affixed to the patient's body. Arms can be uniformly spaced about attachment portion 26 to balance a pulling force applied in multiple directions. In some embodiments, it may be desirable to limit the number of arms to no more than three to provide space for cables that may extend from the wearable medical sensor. However, more than three arms can be provided and may be preferred in some applications. Adjacent arms 28 can be separated as shown in FIG. 1 and free to separately move (e.g., up and down) to conform to a patient's body upon deployment.

Arms 28 can include end bodies 38 disposed at an end of arms 28 opposite attachment portion 26. End bodies 38 include adhesive layer 30 on bottom side 24 and are configured to affix adherable anchor 12 to the patient's body. End bodies 38 can have a planar surface. End bodies can have any shape suitable for securing the wearable medical sensor to the patient's body. End bodies 38 and thereby adhesive layer 30 can have a surface area designed to provide long-term adhesion that is not compromised by movement of the patient's body. End bodies 38 can extend outward from sides of arms 28 and such that end bodies 38 are wider than arms 28 as shown in FIG. 1. In some embodiments, adjacent end bodies 38 can be connected to form a single adhesive end body that extends fully around a periphery of fastening device 10 as shown in phantom in FIG. 1. Any number of arms 28 can extend from attachment portion 26 to the single end body. Arms 28 can have a rigidity sufficient to maintain the of end bodies 38. Edges of end bodies 38 can be rounded to limit irritation to the skin and to provide a more comfortable fit.

Adhesive layer 30 is disposed on bottom side 24 of end bodies 38. Adhesive layer 30 can fully cover bottom side 24 of end bodies 38. Adhesive layer 30 can include one or more layers of material, including different adhesive materials. Adhesive layer 30 includes a biocompatible adhesive suitable for adhesion to skin of a patient and capable of providing a desired adhesive strength for long-term monitoring. Adhesive layer 30 can include, for example 3M™ Medical Tape 1527ENP (single-coated medical plastic tape). Adhesive layer 30 can additionally include an adhesive material layer suitable for bonding the biocompatible adhesive material to bottom side 24 of end body 38. Suitable adhesives can include, for example, 3M™ 200MP double-sided PSA. In some embodiments, a third, interface, layer can be provided between adhesive materials.

In some embodiments, arms 28 can include one or more cutouts where material is absent extending between attachment portion 26 and end bodies 38. Cutouts can provide arms 28 with additional flexibility to conform to a patient's body. Cutouts can have any shape suitable for providing a desired flexibility.

As shown in FIG. 1, cutouts can be defined by a plurality of struts 44 connecting attachment portion 26 to end bodies 38. Each arm 28 can have two or more struts 44. Struts 44 can vary in a width dimension between attachment portion 26 and end bodies 38. For example, a width of struts 44 adjacent to attachment portion 26 can be greater than a width of struts 44 adjacent to end bodies 38. As shown in FIG. 1, struts 44 can extend across an outer perimeter of attachment portion 26 such that struts 44 of adjacent arms 28 are connected at attachment portion 26. The shape, width, number, and spacing of struts 44 can be selected to provide a desired flexibility for arms 28, while maintaining a rigidity of arms 28 sufficient to maintain a position of end bodies 38 relative to attachment portion 26 when external forces (beyond gravity) are removed. A length of arms 28 or struts 44 can be selected based on a width, shape, and thickness of the wearable medical sensor. Generally, arms 28 can be designed to position end bodies 38 adjacent to but outward from a perimeter of the wearable medical sensor on the body of the patient.

Adherable anchor 12 can be formed of a plastic material, including but not limited to a PETG plastic. Adherable anchor 12 can be formed of a clear or transparent material allowing a user to view or monitor the wearable medical sensor. Adherable anchor 12 can have a uniform thickness. For example, adherable anchor 12 can be formed from a 0.03-inch clear PETG plastic. Adherable anchor 12 can be laser cut and cold formed or molded to a predefined shape. As shown in FIG. 2, adherable anchor 12 can be cold formed or molded such that a portion of each arm 28 (i.e., struts 44) extending between attachment portion 26 and end body 38 curves downward between attachment portion 26 and end body 38. Attachment portion 26 and end bodies 38 can be substantially planar with end bodies 38 oriented substantially parallel to attachment portion 26. The curvature of arms 28 can be designed to locate end bodies 38 at desired position relative to a sensor face of the wearable medical sensor as discussed further herein.

Handle 14 extends above top side 22 of adherable anchor 12 and is configured to allow a practitioner to remove release liners 16 from adhesive layer 30 on end bodies 38 in a single motion (i.e., pulling handle upward from the wearable medical sensor and adherable anchor 12). Handle can include junction 20 disposed above attachment portion 26. Junction 20 can be a location at which a plurality of release strips 18 is joined. Release strips 18 can extend from junction 20 to end bodies 38. During deployment, a practitioner can grasp junction 20 to pull handle 14 upward and away from adherable anchor 12. Junction 20 can have any shape, configuration, or additional feature to aid in the deployment of adherable anchor 12. As shown in FIGS. 1 and 2, junction 20 is a ring. In other embodiments, junction 20 can be formed by the intersection of release strips 18. Other configurations are contemplated.

Release strips 18 can be narrow, rectangular, strips as shown in FIGS. 1-3. Release strips 18 extend from junction 20 to inner edge 40 on bottom side 24 of end bodies 38. As such, release strips 18 extend across end bodies 38 from outer edge 42 to inner edge 40. Inner edge 40 is disposed between attachment portion 26 and outer edge 42. Outer edge 42 forms the outermost extent of arms 28. As shown in FIGS. 1-3, release strips 18 can extend from junction 20 to outer edge 42 of end bodies, fold under end bodies 38 at outer edge 42, and extend to inner edge 40.

Release strips 18 connect to release liners 16 at inner edge 40. Release liners 16 are disposed on adhesive layer 30 of end bodies 38. Each release liner 16 covers adhesive layer 30 on end body 38. Release liners 16 can be shaped to match a shape of end bodies 38. Each release liner 16 can extend from inner edge 40 to outer edge 42 of end body 38. Each release strip 18 can be connected to release liner 16 adjacent to inner edge 40. As shown in FIGS. 1-3, release strips 18 can fold at inner edge 40 to form release liners 16. Release liners 16 are formed of a material capable of separating from adhesive layer 30 cleanly or without removing adhesive layer 30.

Handle 14 can be a unitary body with all portions of handle 14 formed of a material suitable for serving as a release liner. Handle 14 can be formed, for example, from a silicone coated PETG plastic.

FIG. 4 is a perspective side view of fastening device 10 coupled to wearable medical sensor 46. Fastening device 10, adherable anchor 12, handle 14, release liners 16, release strip 18, junction 20, top side 22, bottom side 24, attachment portion 26, arms 28, adhesive layer 30, hole 32, and slits 34, anti-rotation member 36, end bodies 38, inner edge 40, outer edge 42, struts 44, wearable medical sensor 46, sensor head 48, sensor face 50, mounting member 52, cable 54, and skin 56 are shown.

Fastening device 10 can be used to securely attach a variety of wearable medical sensors to a patient's body with ease and is not limited to use with the disclosed wearable medical sensor 46. Wearable medical sensor 46 shown in FIG. 4 is an ultrasound transducer configured for long-term monitoring. Wearable medical sensor 46 includes sensor head 48 having sensor face 50 disposed to contact skin 56 of a patient's body. Sensor head 48 can include mounting member 52 disposed on a surface opposite sensor face 50 and configured for connection with attachment portion 26 of adherable anchor 12. Wearable medical sensor 46 can include one or more cables 54 connecting sensor head 48 to a controller and/or monitoring unit (not shown). As shown in FIG. 4, attachment portion 26 extends across a top surface of sensor head 48 and is secured to sensor head 48 via mounting member 52. Bottom side 24 of attachment portion 26 is disposed adjacent to sensor head 48. Anti-rotation members 36 can be positioned on either side of a junction between cable 54 and sensor head 48 to restrict movement of the transducer 46 relative to adherable anchor 12 and vice versa.

Arms 28 curve around sides of sensor head 48 positioning end bodies 38 below attachment portion 26 and at a height position relative to sensor face 50, which can be equal to, or above sensor face 50. End bodies 38 can be disposed in a plane substantially parallel to attachment portion 26 and separated from attachment portion 26 by a distance that is less than a thickness or height of sensor head 46 such that end bodies 38 are disposed above sensor face 50. Sensor face 50 can be disposed in a plane substantially parallel to a plane in which end bodies 38 are disposed. The height position of end bodies 38 relative to sensor face 50 is illustrated as distance d. End bodies 38 secure wearable medical sensor 46 to skin 56. The height position of end bodies 38 relative to sensor face 50 determines a force applied by adherable anchor to wearable medical sensor 46 after deployment and during long-term monitoring. In some applications, it is necessary to exert a force on the wearable medical sensor to ensure complete and continuous contact between sensor face 50 and skin 56. For example, pulse oximetry sensors (SpO₂) sensors work best if sufficient pressure (10-20 mmHg) is applied to push the venous blood out of the tissue volume being sampled. For such applications, adherable anchor 12 can be cold formed or molded to locate end bodies 38 slightly above sensor face 50 a distance determined to provide suitable pressure on wearable medical sensor 46 without compromising adhesion to skin 56. In other applications, it may be desirable to locate end bodies 38 in the same plane as sensor face 50 to securely affix wearable medical sensor 46 to skin 56 without applying significant pressure to wearable medical sensor 46. As discussed further herein, lifting handle 14 causes end bodies 38 to be lifted away from skin 56. When release liners 16 are released, arms 28 can return to their predefined shape, causing end bodies 38 to snap against skin 56.

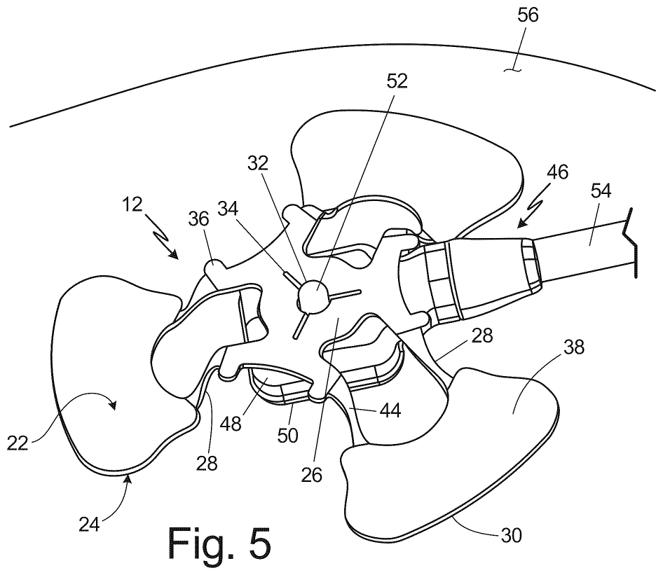

FIG. 5 is a perspective top view adherable anchor 12 coupled to wearable medical sensor 46 following deployment. Adherable anchor 12, top side 22, attachment portion 26, arms 28, adhesive layer 30, hole 32, slits 34, anti-rotation member 36, end bodies 38, struts 44, wearable medical sensor 46, sensor head 48, mounting member 52, cable 54, and skin 56 are shown.

As shown in FIG. 5, adherable device is formed of a transparent material allowing a practitioner to see wearable medical sensor 46 during a long-term monitoring process. Attachment portion 26 extends across a top surface of sensor head 48. Mounting member 52 extends through and is retained by hole 32 of attachment portion 26. Arms 28 curve around sides of sensor head 48 positioning end bodies 38 outward from sensor face 50 on skin 56. Both sensor face 50 and end bodies 38 are secured to skin 56. Adherable anchor 12 is designed such that arms 28 are substantially taut when attached to skin 56. Anti-rotation member 36 can prevent rotation of sensor head 48 after wearable medical sensor 46 has been affixed to skin 56 via adherable anchor 12.

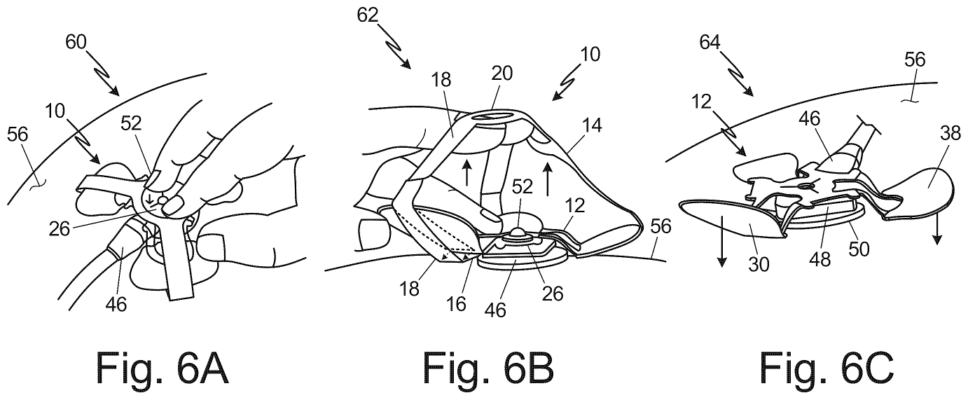

FIGS. 6A-6C are perspective views of steps of a method for affixing wearable medical sensor 46 to a body of a patient with fastening device 10. Fastening device 10, adherable anchor 12, handle 14, release liners 16, release strip 18, junction 20, top side 22, bottom side 24, attachment portion 26, arms 28, adhesive layer 30, hole 32, and slits 34, end bodies 38, inner edge 40, outer edge 42, struts 44, wearable medical sensor 46, sensor head 48, sensor face 50, mounting member 52, and skin 56 are shown.

In step 60, shown in FIG. 6A, fastening device 10 is attached to wearable medical sensor 46. Fastening device 10 can be attached to wearable medical sensor 46 by pressing mounting member 52 of sensor head 48 through hole 32 in attachment portion 26 of adherable anchor. Slits 34 can expand hole 32 to fit mounting member 52. Mounting member 52 can be retained in hole 32. As previously discussed, attachment portion 26 can include other means of attachment in addition to or alternatively to hole 32 and slits 34. For example, attachment portion 26 can include an adhesive on bottom side 24 configured to adhere to sensor head 48. Other mechanical attachment mechanisms known in the art are contemplated. Step 60 can be performed while wearable medical sensor 46 is positioned on skin 56 in the target area of interest for long-term monitoring or fastening device 10 can be secured to wearable medical sensor 46 before wearable medical sensor 46 is applied to skin 56.

In step 62, shown in FIG. 6B, handle 14 is pulled to remove release liners 16 from adhesive layer 30 on adherable anchor 12. As shown, handle 14 can be pulled upward from junction 20 away from attachment portion 26, wearable medical sensor 46, and the patient's body. Generally, a practitioner can fix the position of wearable medical sensor 46 on skin 56 with one hand (e.g., by pressing on sensor head 48 and thereby attachment portion 26) while pulling handle 14 with the other hand. In some cases, it may be feasible for a practitioner to secure wearable medical sensor 46 and pull handle 14 with the same hand.

As handle 14 is pulled upward and away from attachment portion 26, release strips 18 pull release liners 16 from inner edge 40 of end bodies 38. This motion can cause arms 28 and end bodies 38 to flex upward away from skin 56 as shown in FIG. 6B. Because handle 14 is attached to all release liners 16, pulling handle 14 straight upward can cause release strips 18 to remove all release liners 16 simultaneously.

In step 64, shown in FIG. 6C, end bodies 38, which were lifted above skin 56 when handle 14 was being pulled, can snap against skin 56 as arms 28 return to their predefined shape. The exposed adhesive layer 30 on end bodies 38 contacts skin 56 and affixes wearable medical sensor 46 to skin 56. Depending on the starting position of end bodies 38 relative to sensor face 50, little or no additional pressure may need to be applied by a practitioner to end bodies 38 to secure attachment. Although, a practitioner may press against each end body 38 to ensure it is securely affixed to skin 56.

Once long-term monitoring is complete, wearable medical sensor 46 can be removed by removing end bodies 38 from skin 56. End bodies 38 can typically be released by pulling from edges of end bodies 38.

The disclosed fastening device can be used to affix a variety of wearable medical sensors easily and securely to a patient's body for long-term monitoring. The disclosed fastening device can be adapted for use with wearable medical sensors of a variety of shapes and sizes and that require varying degrees of downward applied pressure to ensure proper functioning. The embodiments disclosed herein are intended to provide an explanation of the present invention and not a limitation of the invention. The present invention is not limited to the embodiments disclosed. It will be understood by one skilled in the art that various modifications and variations can be made to the invention without departing from the scope and spirit of the invention.

In one embodiment, wearable medical sensor 46 can be an ultrasound transducer array configured for measuring doppler flow signals of blood flow to organs in the abdomen of the patient, such as (but not limited to) the kidneys, the liver, the pancreas, and the spleen. In order to detect and measure the doppler flow signals of blood flow to an organ in the abdomen of the patient, such as the kidneys, the elements of the ultrasound transducer array can have a low operating frequency between 0.5 MHz and 4.0 MHz and a size greater than one wavelength in soft tissue. With an operating frequency between 0.5 MHz and 4.0 MHz and an element size greater than one wavelength in soft tissue, the ultrasound transducer array can produce ultrasound beams that penetrate more than 15 cm into the patient, which is a sufficient depth to measure renal blood flow, hepatic blood flow, splenic blood flow, and pancreatic blood flow.

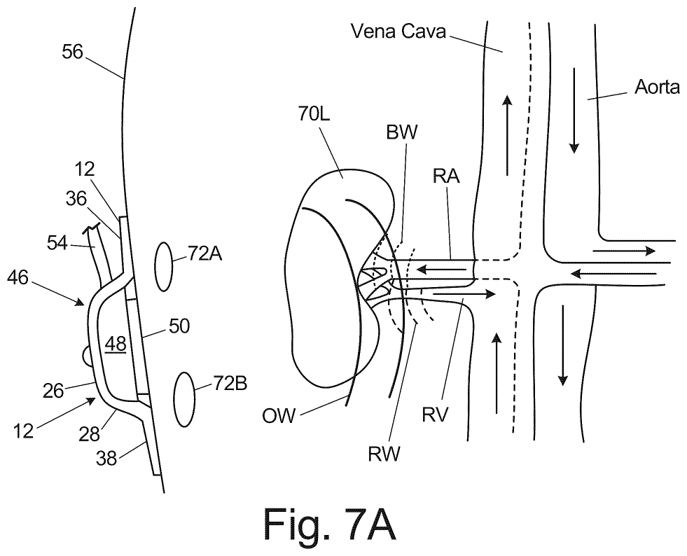

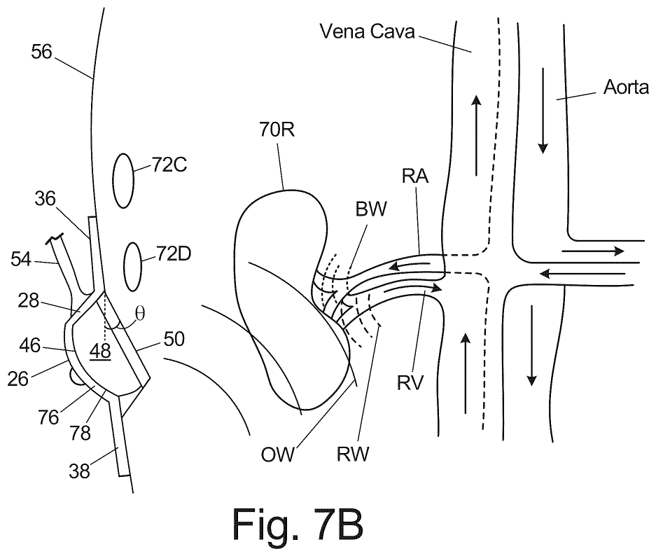

FIG. 7A is a schematic diagram of wearable medical sensor 46 attached to an abdomen of a patient by adherable anchor 12 to monitor the left kidney 70L of a patient. Wearable medical sensor 46 is an ultrasound transducer configured to monitor the doppler flow signal of the left kidney 70L, which can be measured from either the renal artery RA as blood enters the left kidney 70L from the aorta of the patient via the renal artery RA or from the renal vein RV as blood exits the left kidney 70L to the vena cava of the patient via the renal vein RV. FIG. 7A shows originating wave OW from wearable medical sensor 46 and return waves, identified as doppler signals BW of blood flow in the renal artery RA and doppler signal RW of blood flow in the renal vein RV. Due to doppler physics, the doppler signal BW of the blood flow in the renal artery RA is “blue shifted” as the blood flow in the renal artery RA is moving toward the ultrasound transducer probe (sensor head 48 of wearable medical sensor 46). The doppler signal RW of the blood flow in the renal vein RV is “red shifted” as the blood flow in the renal vein RV is moving away from the ultrasound transducer. Since the doppler signal BW is blue shifted and the doppler signal RW is red shifted, wearable medical sensor 46 can easily distinguish renal artery blood flow from renal vein blood flow. In human subjects, the renal artery RA and renal vein RV are close and aligned parallel enabling wearable medical sensor 46 to capture both arterial and venous flow of the left kidney 70L simultaneously.

As illustrated in FIG. 7A, sensor face 50 of wearable medical sensor 46 is positioned flat on the abdomen of the patient over the left kidney 70L and over at least one intercostal space between adjacent ribs 72A, 72B. As illustrated, sensor face 50 is oriented approximately perpendicular to renal artery RA and renal vein RV to direct the originating wave OW substantially parallel to the blood flow in the renal artery RA and renal vein RV. Adherable anchor 12, which includes attachment portion 26, arms 28, and end bodies 38, extends over a top of sensor head 48 to secure sensor head 48 to skin 56 as previously described. Arms 28, which connect attachment portion 26 to end bodies 38 are equal in length and thereby cause adherable anchor 12 to substantially evenly distribute a pressure across sensor head 48 and the abdomen of the patient.

In some applications, it may be necessary to apply increased force to one or more locations of wearable medical sensor 46 offset from a center to tilt sensor face 50 toward an area of interest (e.g., kidney). For example, it may be possible to obtain improved doppler signals in ultrasonic monitoring of kidney perfusion by positioning sensor face 50 below the ribs and tilting sensor face 50 upward toward the kidney. This orientation may be particularly useful in monitoring the right kidney 70R as shown in FIG. 7B due to a slight bend observed in the renal artery RA and renal vein RV of the right kidney 70R.

FIG. 7B is a schematic diagram of wearable medical sensor 46 attached by adherable anchor 76 to the side or flank of a patient below ribs 72C, 72D to monitor perfusion of the right kidney 70R. As discussed with respect to FIG. 7A, wearable medical sensor 46 can be an ultrasound transducer configured to monitor the doppler flow signal of the right kidney 70R, which can be measured from either the renal artery RA as blood enters the right kidney 70R from the aorta of the patient via the renal artery RA or from the renal vein RV as blood exits the right kidney 70R to the vena cava of the patient via the renal vein RV.

As illustrated in FIG. 7B, adherable anchor 76 is configured to apply increased pressure to a top surface of end 78 (opposite cable 54) of sensor head 48, causing sensor head 48 to press inward on the patient's body and orient sensor face 50 toward the right kidney 70R and approximately perpendicular to renal artery RA and renal vein RV. FIG. 7B shows tilt angle θ, which is the degree to which sensor face 50 is tilted relative to the nominal surface (i.e., flat orientation in FIG. 7A). The appropriate or optimal tilt angle θ can be determined by observing the signals received by wearable medical sensor 46 in real time. The tilt angle θ can vary depending on the application and the patient's anatomy. For long-term monitoring applications, it may be desirable to minimize the tilt angle θ to limit injury to the patient. For example, the tilt angle θ may be limited to about 30 degrees in most applications.

As described further herein, additional pressure can be applied to any location on the top surface of sensor head 48 offset from center to cause tiling of sensor face 50. It will be recognized that cable 54 may limit the amount of tilting that can be achieved by applying increased pressure at the surface of sensor head 48 adjacent to cable 54. As such, it may be preferrable, when the option is available, to apply increased pressure to end 78 of sensor head 48, which is located opposite cable 54.

Adherable anchor 76 can be substantially similar to adherable anchor 12 with one or more additional and/or alternative features disclosed herein to tilt and maintain the tilt of wearable medical sensor 46. Adherable anchor 76 can be any one of multiple adherable anchor embodiments disclosed herein or combinations thereof configured to apply and maintain a tilt of sensor face 50 toward an area of interest for long-term monitoring.

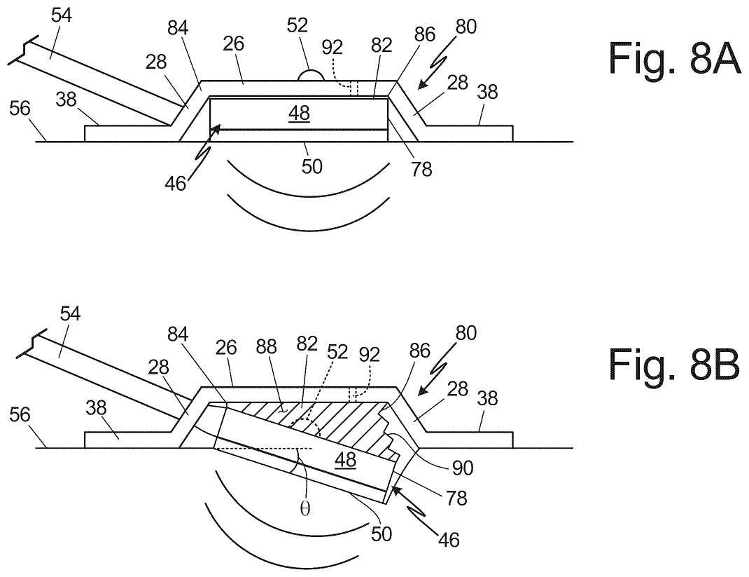

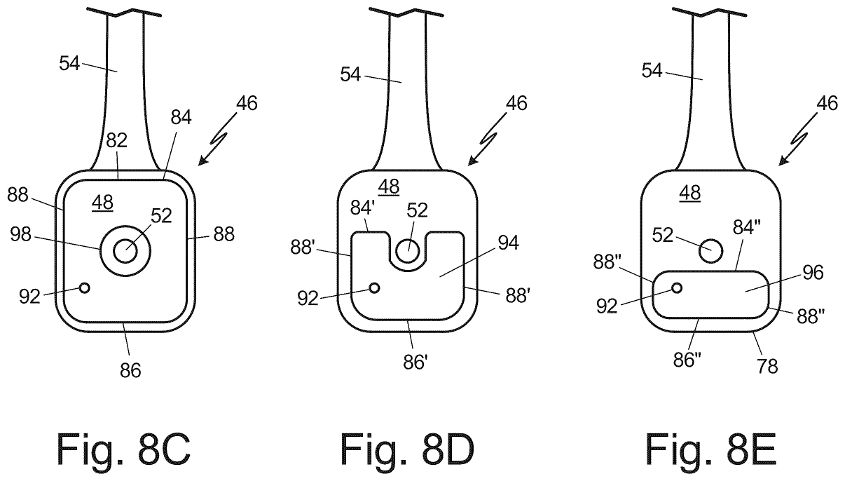

FIGS. 8A and 8B are schematic diagrams of adherable anchor 80 applied to wearable medical sensor 46 and skin 56 of a patient. Wearable medical sensor 46, including sensor head 48, sensor face 50, mounting member 52, and cable 54 are shown. It will be understood by one of ordinary skill in the art that adherable anchor 80 can be applied to or adapted for use with wearable medical sensors of different shapes and/or configurations and is not limited to the embodiment shown.

FIG. 8A shows wearable medical sensor 46 in a substantially flat orientation on the body of the patient with pressure from adherable anchor 80 generally evenly distributed across sensor head 48. FIG. 8B shows wearable medical sensor 46 in a tilted orientation with increased pressure applied to end 78 of wearable medical sensor 46, as described with respect to FIG. 7B. End 78 presses into the body of the patient causing sensor face 50 to tilt with respect to the nominal surface (i.e., flat orientation in FIG. 7A) by tilt angle θ. It will be recognized by one of ordinary skill in the art that the body of a patient is generally not planar and that the tilt angles θ described herein are an approximation. Additionally, several of the embodiments disclosed herein can allow for tilting about multiple axes.

Adherable anchor 80 can be substantially similar to adherable anchor 12 with the addition of inflatable bladder 82. For simplicity, only attachment portion 26, arms 28, end bodies 38, and inflatable bladder 82 are shown. Inflatable bladder 82 is disposed on an underside of attachment portion 26 such that inflatable bladder 82 is positioned between attachment portion 26 and sensor head 48 when adherable anchor 80 is assembled with wearable medical sensor 46. Inflatable bladder 82 can be securely attached to the underside of attachment portion 26, for example, by an adhesive.

Adherable anchor 80 can be used with inflatable bladder 82 in a deflated state as illustrated in FIG. 8A or with any amount of inflation as needed to adjust sensor face 50 to a desired tilt angle θ. FIG. 8B shows inflatable bladder 82 in a fully inflated state.

Inflatable bladder 82 can be formed of a semi-rigid plastic that is collapsible with deflation but resistant to stretching with inflation. Inflatable bladder 82 can have an asymmetric design configured to allow for asymmetric expansion with increased expansion adjacent to end 78. As illustrated in FIG. 8B, inflatable bladder 82 extends from end 84 to opposite end 86 and between side walls 88 (one shown). End 86 is positioned adjacent to end 78 of sensor head 48 and can include an accordion fold or bellows 90. Bellows 90 allow end 86 to expand to varying heights with inflation and to collapse to the substantially flat configuration shown in FIG. 8A with deflation. Side walls 88 can taper in height from end 84 to end 86 and may also include an accordion fold or bellows to allow side walls 88 to expand with inflation and collapse to the substantially flat configuration with deflation. The number of folds or bellows can be selected to reduce the height of end 86 in the collapsed configuration such that a thickness or height of inflatable bladder 82 is substantially uniform from end 84 to end 86 in the collapsed configuration.

Inflatable bladder 82 can be inflated via port 92 opening to an interior cavity of inflatable bladder 82. Port 92 can be provided through attachment portion 26 as shown in FIGS. 8A and 8B or adjacent to attachment portion 26. Preferably, port 92 opens to a top of adherable anchor 80 or inflatable bladder 82 for case of access, although port 92 can be located in any area of inflatable bladder 82 that is accessible for inflating and deflating inflatable bladder 82.

Inflatable bladder can be inflated with any suitable fluid (e.g., air, compressed gas, biocompatible liquid). Port 92 can include a valve (not shown) to prevent release of fluid from inflatable bladder 82. Port 92 can be configured to receive, for example, a needle for filling inflatable bladder 82 by syringe. Fluid can be supplied to inflatable bladder 82 until sensor face 50 is oriented at the desired tilt angle θ. Use of inflatable bladder 82 allows a practitioner to fine tune the tilt angle θ of sensor face 50 based on sensor signals observed in real time.

FIG. 8C is a top view of inflatable bladder 82 positioned on wearable medical sensor 46. FIGS. 8D and 8E show alternative configurations of inflatable bladder 82, including inflatable bladders 94 and 96. Remaining features of adherable anchor 80 are omitted from view. Inflatable bladder 82 can have a ring shape with opening 98 provided to accommodate mounting member 52. Mounting member 52 can be used to locate adherable anchor 80 on wearable medical sensor 46 as described with respect to adherable anchor 12. Once adherable anchor 80 is secured to skin 56 as previously disclosed, mounting member 52 may not be necessary to retain a position of wearable medical sensor 46 relative to adherable anchor 80. As such, upon expansion of inflatable bladder 82, mounting member 52 may be released and displaced from adherable anchor 80, as shown in FIG. 8B. In some embodiments, one or more retention features (not shown) can be provided, for example, on an underside of attachment portion 26, to maintain a position of sensor head when mounting member 52 is released from attachment portion 26. The one or more retention features can additionally help maintain the position of sensor head 48 during long-term monitoring. Retention features may include, for example, ridges or projecting features configured to capture sensor head 48.

In other embodiments, inflatable bladder 82 can be adhered to sensor head 48 by a removable and/or repositionable adhesive to locate adhesive anchor 80 on sensor head 48 and to maintain alignment of inflatable bladder 82 with sensor head 48 upon inflation. In some embodiments, one or more additional retention features (not shown) can be provided on an underside of adherable anchor 80 (e.g., underside of attachment portion 26) to prevent sensor head 48 from slipping or to maintain a position of inflatable bladder 82 relative to sensor head 48 during inflation.

In the embodiment shown in FIGS. 8B and 8C, bellows 90 are located at end 86 of inflatable bladder 82, opposite cable 54 as this end is least restricted from movement by cable 54. In other embodiments, bellows 90 can be located on either side 88 or on end 84, although it may be preferrable to avoid end 84 unless necessary for a particular application in which access is limited.

FIGS. 8D and 8E are top views of alternative inflatable bladder configurations for application with adherable anchor 80. As shown in FIG. 8D, adherable anchor 80 can include inflatable bladder 94, which covers only a portion of the top surface of sensor head 96. Inflatable bladder 94 has a U-shape to accommodate mounting member 52. Inflatable bladder 94 can have substantially the same configuration as inflatable bladder 82 with bellows 90′ located at end 86′ and side walls 88′ that taper in height, and which can also include folds or bellows for collapsing inflatable bladder 94.

As shown in FIG. 8E, adherable anchor 80 can include inflatable bladder 96, which can be positioned on one end or side (e.g., end 78) of the top surface of sensor head 48. To accommodate expansion, an entire perimeter (both sides 88″ and ends 84″ and 86″) of inflatable bladder 96 can include bellows 90″. Inflatable bladders 94 and 96 cover only a portion of the top surface of sensor head 48. As such, a gap may form between attachment portion 26 of adherable anchor 80 and sensor head 48 in the region not covered by inflatable bladder 94 and 96 during inflation.

Adherable anchor 80 can replace adherable anchor 12 in fastening device 10 (shown in FIGS. 1-5 and 6A-6C). As previously described, fastening device 10 (including adherable anchor 80) can be applied to wearable medical sensor 46 when wearable medical sensor 46 is positioned on skin 56 in the target area of interest for long-term monitoring. The practitioner can position fastening device 10 on wearable medical device 46 in an orientation corresponding with the tilt of sensor head 48 (i.e., in which bellows 90 are positioned over the portion of sensor head 48 that is pressed inward). Alternatively, fastening device 10 can be secured to wearable medical sensor 46 before wearable medical sensor 46 is applied to skin 56. A practitioner can position sensor head 48 in the target area of interest with the suitable tilt angle θ before attaching adherable anchor 80 to skin 56. The practitioner can orient sensor head 48 to ensure increased pressure needed for tiling sensor face 50 is applied to end 78, corresponding with the region of inflatable bladder 82, 94, or 96 where expansion is greatest. Once adherable anchor 80 is secured to skin 56, the practitioner can inflate inflatable bladder 82, 94, or 96 to reach the desired tilt angle θ.

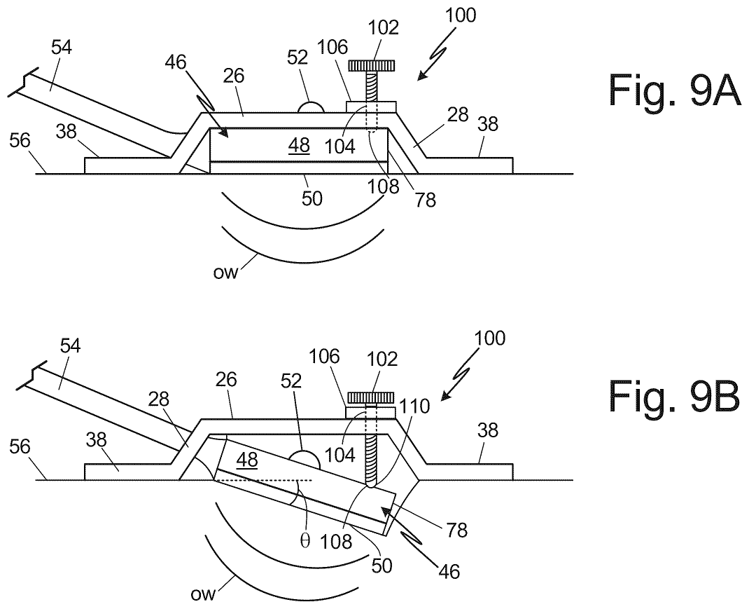

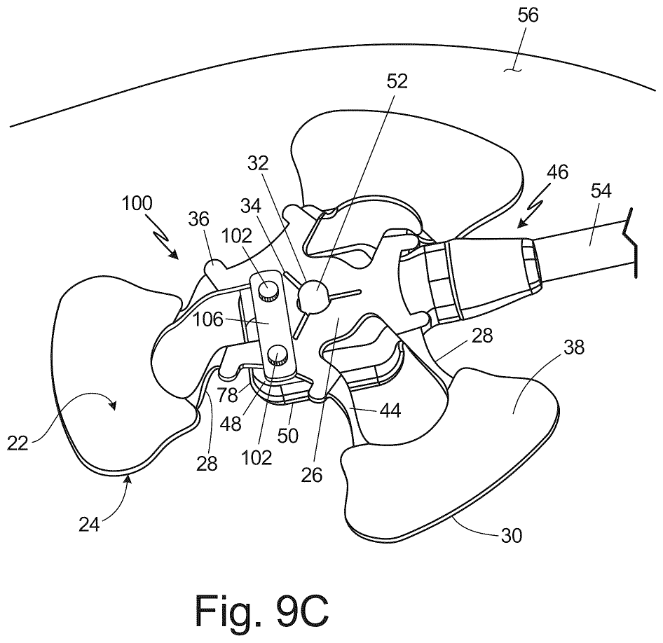

FIGS. 9A and 9B are schematic diagrams of adherable anchor 100 applied to wearable medical sensor 46 and skin 56 of a patient. Adherable anchor 100 can be substantially similar to adherable anchor 80 previously described in reference to FIGS. 8A-8B. In place of inflatable bladder 82 of adherable anchor 80, adherable anchor 100 includes one or more threaded fasteners 102 disposed offset from a center of sensor head 48 (e.g., adjacent to end 78 of sensor head 48) to adjust the tilt angle θ of sensor face 50. The one or more threaded fasteners 102 can be received in threaded holes 104 of rigid member 106. FIG. 9C is a top view illustration of adherable anchor 100 applied to wearable medical sensor 46.

Rigid member 106 can be, for example, a rigid plate secured to attachment portion 26 of adherable anchor and configured to retain a position of one or more threaded fasteners 102 on adherable anchor 100 and sensor head 48. Threaded fasteners 102 can be, for example, set screws that can be turned by hand and/or by screwdriver for adjustment. As illustrated in FIGS. 9A-9C, rigid member 106 can be a rectangular plate positioned over end 78 of sensor head 48. In alternative embodiments, rigid member 106 can be any shape including, for example, a ring circumscribing mounting member 52, to accommodate threaded fasteners 102 at different locations on the top surface of sensor head 48. Rigid member 106 can include a plurality of threaded holes 104 (not shown) configured to receive threaded fasteners 102 enabling a practitioner to select the one or more locations on which to apply additional pressure to sensor head 48 to achieve a desired tilt of sensor face 50.

Sensor head 48 can include one or more dimples or depressions 108 configured to locate ends 110 of the one or more threaded fasteners 102 and prevent the one or more threaded fasteners 102 from slipping out of place during adjustment and long-term monitoring. One or more depressions 108 can be, for example, circular or semi-spherical in shape to match a shape of ends 110 or can be a trough configured to capture multiple ends 110 of threaded fasteners 102. Ends 110 of threaded fasteners 102 can be rounded to prevent damage to sensor head 48.

As illustrated in FIG. 9C, two threaded fasteners 102 can be positioned adjacent to end 78 of sensor head 48 opposite cable 54. In alternative embodiments, a single threaded fastener 102 or more than two threaded fasteners 102 can be used. A single threaded fastener 102 can be centrally located on an end or side of the top surface of sensor head 48 (e.g., end 78) to distribute pressure uniformly across the end or side, can be located on a corner between adjoining ends or sides, or can be located in any other location to obtain a desired tilting of sensor face 50. It will be understood by one of ordinary skill in the art that the number and location of threaded fasteners 102 can be modified based on the size and shape of sensor head 48.

Adherable anchor 100 can replace adherable anchor 12 in fastening device 10. As previously described, fastening device 10 (including adherable anchor 100) can be applied to wearable medical sensor 46 when wearable medical sensor 46 is positioned on skin 56 in the target area of interest for long-term monitoring. The practitioner can position fastening device 10 on wearable medical device 46 in an orientation corresponding with the tilt of sensor head 48 (i.e., in which threaded fasteners 102 or threaded holes 104 are positioned over the portion of sensor head 48 that is pressed inward). Alternatively, fastening device 10 can be secured to wearable medical sensor 46 before wearable medical sensor 46 is applied to skin 56. A practitioner can position sensor head 48 in the target area of interest with the suitable tilt angle θ while fastening device 10 is secured to wearable medical sensor 46 but before attaching adherable anchor 100 to skin 56. The practitioner can orient sensor head 48 to ensure increased pressure needed for tilting sensor head 48 is applied to a portion of sensor head 48 corresponding with the location of the one or more threaded fasteners 102 or threaded holes 104. Once adherable anchor 100 is secured to skin 56, the practitioner can turn the one or more threaded fasteners 102 to press a portion of sensor head 46 into the body of the patient until the desired tilt angle θ of sensor face 50 is reached. The use of one or more threaded fasteners 102 allows the practitioner to fine tune the tilt angle θ of sensor face 50 based on sensor signals observed in real time.

As the one or more threaded fasteners 102 are threaded further into rigid member 106, attachment portion 26 (including rigid member 106) is displaced from sensor head 48 at least in the region of the one or more threaded fasteners 102. Mounting member 52 of sensor head 48 can be released from attachment portion 26 of adherable anchor 100 as the tilt angle θ is increased. One or more retention features (not shown) can be provided, for example, on an underside of attachment portion 26, to maintain a position of sensor head when mounting member 52 is released from attachment portion 26. The one or more retention features can additionally help maintain the position of sensor head 48 during long-term monitoring. Retention features may include, for example, ridges or projecting features configured to capture sensor head 48.

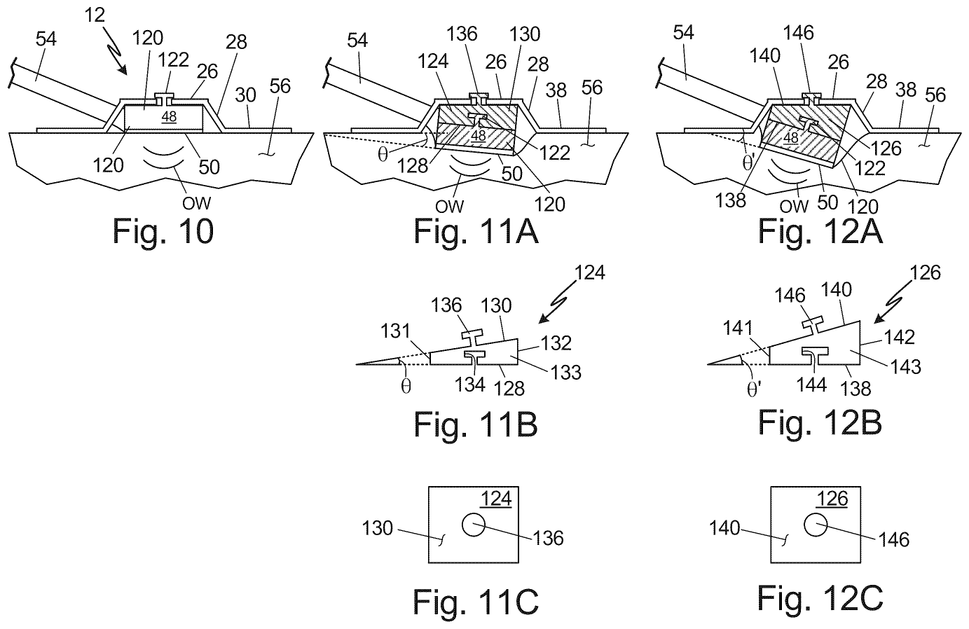

FIG. 10 is a schematic diagram of adherable anchor 12 applied to wearable medical sensor 120 and skin 56 of a patient. Wearable medical sensor 120 is substantially the same as wearable medical sensor 46, having sensor head 48, sensor face 50, and cable 54. Wearable medical sensor 120 can have a modified mounting member 122 to accommodate angled blocks used to adjust the tilt angle θ of sensor face 50 for long-term monitoring as shown in 11A and 11B. In other embodiments, mounting member 122 can be substantially the same as mounting member 52 of wearable medical sensor 46. Mounting member 122 can be configured to retain attachment portion 26 of adherable anchor 12 as shown in FIG. 10 when wearable medical sensor 120 is disposed in a flat or non-angled orientation. In alternative embodiments, wearable medical sensor 120 can be used with a flat block (not shown) of uniform thickness disposed thereupon and having a mounting member shaped to retain adherable anchor 12. FIG. 10 shows wearable medical sensor 120 in a substantially flat orientation with adherable anchor 12 applying substantially uniform pressure across sensor head 48 as described with respect to FIG. 7A.

FIG. 11A is a schematic cross-sectional diagram of adherable anchor 12 applied to wearable medical sensor 120 and skin 56 with the addition of block 124 configured to orient sensor face 50 at a first tilt angle θ. FIGS. 11B and 11C are side and top views of block 124, respectively. FIG. 12A is a schematic cross-sectional diagram of adherable anchor 12 applied to wearable medical sensor 120 and skin 56 with the addition of block 126 configured to orient sensor face 50 at a second tilt angle θ′ greater than the first tilt angle θ. FIGS. 12B and 12C are side and top views of block 126, respectively. FIGS. 11A-11C and 12A-12C are discussed together.

Each of blocks 124 and 126 is configured to attach to mounting member 122 of sensor head 48 and to adherable anchor 12. Blocks 124 and 126 are interchangeable and determine the degree to which sensor face 50 is tilted (i.e., tilt angle θ and tilt angle θ′, respectively). Blocks 124 and 126 can be generally wedge-shaped. Block 124 includes bottom wall 128, top wall 130, opposing end walls 131, 132, opposing side walls 133, slot or receptacle 134, and mounting member 136. Bottom wall 128 is substantially flat with slot or receptacle 134 provided therein and opening to bottom wall 128. Top wall 130 is angled with respect to bottom wall 128 and includes mounting member 136 projecting therefrom. Opposing end walls 131, 132 connect bottom and top walls 128, 130 and are of unequal heights. Opposing side walls 133 connect bottom and top walls 128, 130 and opposing end walls 131, 132 and taper in height from end wall 131 to end wall 132. Block 126 includes bottom wall 138, top wall 140, opposing end walls 141, 142, opposing side walls 143, slot or receptacle 144, and mounting member 146. Bottom wall 138 is substantially flat with slot or receptacle 144 provided therein and open to bottom wall 138. Top wall 140 is angled with respect to bottom wall 138 and includes mounting member 146 projecting therefrom. Opposing end walls 141, 142 connect bottom and top walls 138, 140 and are of unequal heights. Opposing side walls 143 connect bottom and top walls 138, 140 and opposing end walls 141, 142 and taper in height from end wall 141 to end wall 142. Bottom walls 128 and 138, slots 134 and 144, and mounting members 136 and 146 can be substantially the same. Slots 134 and 144 are configured to receive mounting member 122 of wearable medical sensor 120. Mounting members 136 and 146 are configured to be received by attachment portion 26 of adherable anchor 12.

Blocks 124 and 126 are two examples of a plurality of wedge shaped blocks that can be used with wearable medical sensor 120 and adherable anchor 12 to tilt sensor face 50 toward an area of interest. Each block can be configured to provide a specific tilt angle θ for sensor face 50 defined by the angle of the top wall relative to the bottom wall. As illustrated in FIG. 11B, top wall 130 of block 124 is angled with respect to bottom wall 128 by the first tilt angle. As illustrated in FIG. 12B, top wall 140 of block 126 is angled with respect to bottom wall 138 by the second tilt angle θ′, which is greater than first tilt angle θ.

As illustrated in FIGS. 11A and 12A, mounting member 122 of wearable medical sensor 120 can be a projecting member having a nail-like shape with a shank portion extending from sensor head 48 and wider head at an outermost end. Slots 134 and 144 can be shaped and sized for case of assembly with mounting member 122 and retention of mounting member 122. Slots 134 and 144 can have a shape that substantially matches the shape of mounting member 122. Slots 134 and 144 can extend through a full width (between side walls 133) of blocks 124, 126 as shown in FIGS. 11B and 12B or can extend through a partial width of blocks 124, 126. Slots 134 and 144 can be configured to receive mounting member 122 by sliding blocks 124, 126 across a top surface of sensor head 48. In some embodiments, slot walls can taper to provide a friction fit with mounting member 122 when disposed in a fully assembled position. In alternative embodiments, slots 134 and 144 can be configured to attach to mounting member 122 from above. For example, slots 134 and 144 can have a keyhole fastener configuration in which mounting member 122 is received in slots 134, 144 from below and slid into a locked position as known in the art. In some embodiments, mounting member 122 can be substantially the same as mounting member 52 of wearable medical device 46 and slots 134, 144 can have a geometry substantially matching a geometry of mounting member 122 configured to receive mounting member 122. The size, shape, and location of mounting member 122 and slots 134, 144 is not limited to the embodiments shown or described herein. Alternative configurations that are capable of locating and retaining block 124 or 126 on wearable medical sensor 120 for long-term monitoring are contemplated though not fully described herein.

Mounting members 136 and 146 are configured to be received in attachment portion 26 of adherable anchor 12 as described with respect to mounting member 52 of wearable medical sensor 46 shown in FIG. 4. Mounting members 136 and 146 can be substantially the same as mounting member 52 of wearable medical sensor 46. Mounting members 136 and 146 extend perpendicular to top walls 130 and 140, respectively such that mounting members 136 and 146 are oriented perpendicular to attachment portion 26 when deployed. As illustrated in FIGS. 11C and 12C, mounting members 136 and 146 can be centrally located on top walls 130 and 140, respectively.

Blocks 124 and 126 can have a shape and size substantially matching a shape of sensor head 48 such that bottom walls 128 and 138 substantially cover sensor head 48.

Multiple blocks of varying tilt angles can be available for use with adherable anchor 12 and wearable medical sensor 120. For example, blocks can be available in sizes that increase in tilt angle in 5 degree increments (e.g., 5°, 10°, 15° . . . 30°). Smaller increments can be provided to provide finer tuning of the tilt angle θ, however, coarse tuning can be sufficient in many applications.

While blocks 124 and 126 provide tilting about a single axis, alternative blocks that include opposing side walls 133 that vary in height from each other can provide tilting about a second axis.

Fastening device 10 (including adherable anchor 12) can applied with a block of desired tilt angle (e.g., block 124 or 126) to wearable medical sensor 120 when wearable medical sensor 120 is positioned on skin 56 in the target area of interest for long-term monitoring. A practitioner can position sensor head 48 in the target area of interest with the suitable tilt angle θ. Based on the observed tilt of sensor head 48, the practitioner can determine which block is appropriate for maintaining the desired tilt angle. The selected block can be attached to sensor head 48 while sensor head 48 is positioned on the target area of interest. Adherable anchor 12 can then be secured to the block (e.g., via mounting member 136 or 146) and secured to skin 56 of the patient for long-term monitoring. Alternatively, fastening device 10 and a selected block can be secured to wearable medical sensor 46 before wearable medical sensor 46 is applied to skin 56. A practitioner can position sensor head 48 in the target area of interest while fastening device 10 and the selected block are secured to wearable medical sensor 46 but before attaching adherable anchor 12 to skin 56.

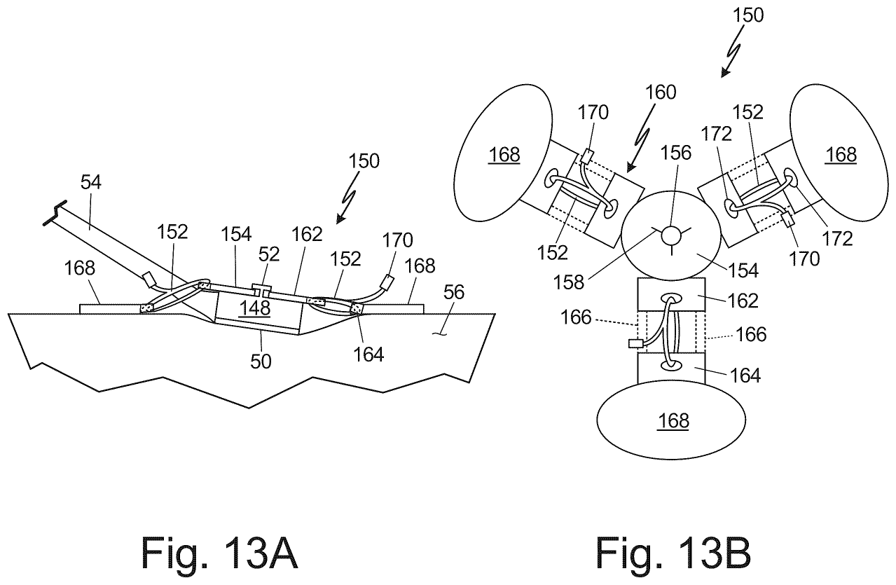

FIG. 13A is a schematic diagram of adherable anchor 150 applied to wearable medical sensor 46 and skin 56 of a patient. Sensor head 48, sensor face 50, mounting member 52, and cable 54 of wearable medical sensor 46 are shown. Adherable anchor 150 can be substantially similar to adherable anchor 12 with the addition of cinching members 152 configured to tighten one or more arms of adherable anchor 150 to tilt sensor face 50 toward a target area of interest. FIG. 13B is a schematic top view of adherable anchor 150. FIG. 13B shows attachment portion 154 with hole 156 and slits 158; arms 160 with inner and outer wings 162, 164, optional struts 166 (shown in phantom), cinching members 152, and end bodies 168. Adherable anchor 150 can include other features of adherable anchor 12, including but not limited to anti-rotation features, which are not shown for simplicity. FIGS. 13A and 13B are discussed together herein.

Attachment portion 154 and arms 160 can be substantially similar to attachment portion 26 and arms 28, respectively, of adherable anchor 12, with the addition of cinching members 152 and inner and outer wings 162, 164 to arms 160. Inner and outer wings 162, 164 can provide attachment points for cinching members 152. Inner wings 162 extend from attachment portion 154. Outer wings extend from end body 168. Cinching members 152 connect inner and outer wings 162 and 164.

Adherable anchor 150 can be configured for use with handle 14 as described with respect to fastening device 10. In some embodiments, cinching members 152 can be formed of a rigid material capable of maintaining a shape of adherable anchor 150 and/or position of end bodies 168 during deployment as described with respect to adherable anchor 12. In some embodiments, arms 160 can include struts 166. Struts 166 can be substantially the same as struts 44 of adherable anchor 12 and can be configured to provide increased rigidity to adherable anchor 150 to ensure that adherable anchor 150 returns to a preset shape when handle 14 is removed during application to a body of a patient.

Cinching members 152 can be adjustable tie wrap-style straps with teeth that lock a position. Cinching members 152 can include ends 170, which can be pulled by a practitioner to tighten cinching members 152. As illustrated in FIG. 13B, cinching members 152 can be threaded through holes 172 in inner and outer wings 162, 164 with ends 170 fed through a connecting receptacle on an opposing end to form a ring as known in the art. In alternative embodiments, one of inner wing 162 and outer wing 164 can include a cinching strap (e.g., attached to or co-molded with inner or outer wing 162, 164) and the other of inner wing 162 and outer wing 164 can include a connecting receptacle (e.g., attached to or co-molded with inner or outer wing 162, 164) configured to capture and lock the cinching strap.

Each arm 160 can include one or more cinching members 152. In some embodiments, each arm 160 can include a single centrally located cinching member 152 with or without struts 166 disposed on either side of cinching member 152. In other embodiments, each arm 160 can include two cinching members 152 positioned, for example, in the location of struts 166.

One or more cinching members 152 can be tightened to adjust a pressure of sensor face 50 on skin 56 of the patient and to adjust the tilt angle θ of sensor face 50. Cinching members 152 allow a practitioner to finely adjust the tilt angle θ of and/or pressure applied by sensor face 50 based on sensor signals observed in real time. Tightening cinching members 152 on multiple arms 160 can allow for tilting of sensor face 50 about multiple axes.

Adherable anchor 150 can replace adherable anchor 12 in fastening device 10. As previously described, fastening device 10 can be applied to wearable medical sensor 46 when wearable medical sensor 46 is positioned on skin 56 in the target area of interest for long-term monitoring. The practitioner can position fastening device 10 on wearable medical device 46 in an orientation corresponding with the tilt of sensor head 48 (i.e., in which cinching members 152 are positioned adjacent to the portion of sensor head 48 that is pressed inward). Alternatively, fastening device 10 can be secured to wearable medical sensor 46 before wearable medical sensor 46 is applied to skin 56. A practitioner can position sensor head 48 in the target area of interest with the suitable tilt angle e while fastening device 10 is secured to wearable medical sensor 46 but before attaching adherable anchor 150 to skin 56. The practitioner can orient sensor head 48 to ensure increased pressure needed for tiling sensor head 48 is applied to an end (i.e., end 78) corresponding with the location of a cinching member 152. Once adherable anchor 150 is secured to skin 56, the practitioner can pull one end 170 of cinching member 152 tighten arm 160 and to press the end (i.e., end 78) of sensor head 46 into the body of the patient until the desired tilt angle θ of sensor face 50 is reached. Other cinching members 152 may also be tightened as needed to retain a position of sensor head 48 for long-term monitoring or to provide tilt about a second axis. Struts 166 (if present) can bend to accommodate cinching of arms 160 with tightening of cinching members 152.

It will be understood by one of ordinary skill in the art that adherable anchor 150 can be applied to or adapted for use with wearable medical sensors of different shapes and/or configurations and is not limited to the embodiments shown and described herein.

The embodiments disclosed herein are intended to provide an explanation of the present invention and not a limitation of the invention. The present invention is not limited to the embodiments disclosed. It will be understood by one skilled in the art that various modifications and variations can be made to the invention without departing from the scope and spirit of the invention. Furthermore, it will be understood by one of ordinary skill in the art that features of one embodiment may be combined with features of another embodiment, though not explicitly described in combination herein.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter or value, without variation, as if no qualifying relative term or term of degree were utilized in the given disclosure or recitation.

### DISCUSSION OF DETAILED EMBODIMENTS

The following are non-exclusive descriptions of possible embodiments of the present invention.

A fastening device is disclosed for attaching a wearable sensor to a body of a patient, the wearable sensor comprising a sensor configured to measure characteristics inside the patient's body. The fastening device includes an adherable anchor configured to affix the wearable sensor to the body of the patient via an adhesive surface and a handle configured to expose the adhesive surface when pulled following attachment of the adherable anchor to the wearable medical sensor. The adherable anchor includes an attachment portion configured to secure the adherable anchor to the wearable medical sensor and an adhesive surface configured to affix the adherable anchor to the body of the patient. The handle is attached to the adhesive surface.

The fastening device of the preceding paragraph can optionally include, additionally and/or alternatively, any one or more of the following features, configurations and/or additional components:

In an embodiment of the foregoing fastening device, the adhesive surface can be disposed on a bottom surface of the adherable anchor, and the handle can extend over a top surface of the adherable anchor and be spaced therefrom.

In an embodiment of any of the foregoing fastening devices, the adherable anchor can include an arm extending outward from the attachment portion. The adhesive surface can be located on the arm.

In an embodiment of any of the foregoing fastening devices, the adherable anchor can include a plurality of arms.

In an embodiment of any of the foregoing fastening devices, each arm of the plurality of arms can include an end body disposed distal to the attachment portion. The adhesive surface can be located on the end body.

In an embodiment of any of the foregoing fastening devices, the handle can include a release liner. The release liner can be disposed on the adhesive surface of the end body and can cover the adhesive surface.

In an embodiment of any of the foregoing fastening devices, the adhesive surface can be located on a first side of the adherable anchor. The handle can extend from the release liner to a second side of the adherable anchor opposite the first side.

In an embodiment of any of the foregoing fastening devices, the handle can include a plurality of strips joined at a junction. Each strip can extend from a release liner disposed on the adhesive surface of the end body.

In an embodiment of any of the foregoing fastening devices, the release liner can extend from a first edge to a second edge of the end body. The first edge can be disposed between the attachment portion and the second edge. Each strip of the plurality of strips can be connected to the release liner adjacent to the first edge.

In an embodiment of any of the foregoing fastening devices, the handle can be configured to remove the release liner in response to pulling of the handle away from the adherable anchor, thereby exposing the adhesive surface of each arm.

In an embodiment of any of the foregoing fastening devices, each arm can include a cutout extending between the attachment portion and the end body.

In an embodiment of any of the foregoing fastening devices, each arm can include a plurality of struts connecting the attachment portion to the end body.

In an embodiment of any of the foregoing fastening devices, the attachment portion and the arms can have a formed shape and can be made of a flexible material configured to return to the formed shape when a force is removed.

In an embodiment of any of the foregoing fastening devices, the attachment portion can be disposed in a first plane and the end body of each arm can be disposed in a second plane that is substantially parallel to the first plane and separated from the first plane by a distance.

In an embodiment of any of the foregoing fastening devices, the distance can be equal to a thickness of the wearable sensor.

In an embodiment of any of the foregoing fastening devices, the distance can be less than a thickness of the wearable sensor.

In an embodiment of any of the foregoing fastening devices, the distance can be less than a thickness of the wearable sensor such that the end bodies are disposed above a sensor face of the wearable sensor upon assembly with the wearable sensor.

In an embodiment of any of the foregoing fastening devices, the attachment portion can include a hole configured to receive a mounting member projecting from a surface of the wearable sensor.

In an embodiment of any of the foregoing fastening devices, the end body can include a first adhesive layer disposed in contact with the end body and a second adhesive layer disposed on the first adhesive layer and forming the adhesive surface.

In an embodiment of any of the foregoing fastening devices, the adherable anchor can include an anti-rotation member configured to retain a position of the wearable sensor with respect to the adherable anchor.

In an embodiment of any of the foregoing fastening devices, the anti-rotation member can include tabs that extend outward from attachment portion.

In an embodiment of any of the foregoing fastening devices, the plurality of arms can include an end body disposed distal to the attachment portion and extending along the periphery of the fastening device, wherein the adhesive surface is located on the end body.

In an embodiment of any of the foregoing fastening devices, the adherable anchor can be formed of a plastic material.

In an embodiment of any of the foregoing fastening devices, the adherable anchor can be transparent.

In an embodiment of any of the foregoing fastening devices, the adherable anchor can include three arms.

In an embodiment of any of the foregoing fastening devices, the arms can be uniformly spaced.

An embodiment of any of the foregoing fastening devices can further include an inflatable bladder disposed under the attachment portion and configured to apply pressure to one end of the wearable sensor toward target area of interest.

An embodiment of any of the foregoing fastening devices can further include a rigid plate disposed on the attachment portion, the rigid plate having a threaded hole, and a threaded fastener received in the threaded hole and configured to apply pressure to the wearable sensor to tilt a sensor face of the wearable sensor toward a target area of interest.

An embodiment of any of the foregoing fastening devices can further include a wedge-shaped block disposed between the attachment portion and the wearable sensor. The wedge-shaped block can be configured to tilt a sensor face of the wearable sensor toward a target area of interest.

A method of attaching a wearable sensor to a body of a patient is disclosed, the wearable sensor comprising a sensor configured to measure characteristics inside the patient's body. The method includes attaching a fastening device to the wearable sensor. The fastening device includes an adherable anchor configured to affix the wearable sensor to skin of the body of the patient and a handle comprising a release liner attached to the adherable anchor. The method further includes positioning the wearable sensor on the body of the patient and pulling a handle of the fastening device to remove the release liner from an adhesive surface of the adherable anchor.

The method of the preceding paragraph can optionally include, additionally and/or alternatively, any one or more of the following features, configurations, additional components, and/or steps:

In an embodiment of the foregoing method, the adherable anchor can include an attachment portion having a hole and wherein attaching the fastening device to the wearable sensor can include pressing a mounting member of the wearable sensor through the hole.

In an embodiment of any of the foregoing methods, the handle can be disposed above the wearable sensor and the adherable anchor and can be pulled upward away from the body of the patient.

In an embodiment of any of the foregoing methods, the adherable anchor can include a central attachment portion configured to secure the adherable anchor to the wearable sensor and a plurality of arms. Each arm of the plurality of arms can extend outward from the central attachment portion and can include an end body separated from the central attachment portion. The end body can include the adhesive surface.

In an embodiment of any of the foregoing methods, pulling the handle upward can cause the plurality of arms to flex and removing the release liner can cause the end bodies to contact the skin of the body of the patient.

In an embodiment of any of the foregoing methods, the adherable anchor can exert a downward force on the wearable sensor toward the body of the patient.

An embodiment of any of the foregoing methods can further include tilting the sensor toward a target area of interest by one of: (1) inflating a bladder disposed between an attachment portion of the adherable anchor and a top surface of the wearable sensor to apply pressure to a portion of the top surface of the wearable sensor, (2) turning a threading a threaded fastener disposed through the adherable anchor and in contact with the top surface of the wearable sensor to apply pressure to a portion of the top surface of the wearable sensor, (3) tightening a cinching member of the adherable anchor to apply pressure to a portion of the top surface of the wearable sensor, and (4) providing a wedge-shaped block between the adherable anchor the top surface of the wearable sensor.

While the invention has been described with reference to an exemplary embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A fastening device for attaching a wearable sensor to a body of a patient, the wearable sensor configured to measure characteristics inside the patient's body, the fastening device comprising:
an adherable anchor configured to affix the wearable sensor to the body of the patient, the adherable anchor comprising:
an attachment portion configured to secure the adherable anchor to the wearable sensor; and
an adhesive surface configured to affix the adherable anchor to the body of the patient; and
a handle attached to the adhesive surface, the handle configured to expose the adhesive surface when pulled following attachment of the adherable anchor to the wearable sensor.

2. The fastening device of claim 1, wherein the adhesive surface is disposed on a bottom surface of the adherable anchor and wherein the handle extends over a top surface of the adherable anchor and is spaced therefrom.

3. The fastening device of claim 1, wherein the adherable anchor comprises a plurality of arms extending outward from the attachment portion.

4. The fastening device of claim 3, wherein each arm of the plurality of arms comprises an end body disposed distal to the attachment portion and wherein the adhesive surface is located on the end body.

5. The fastening device of claim 4, wherein the handle comprises a release liner, the release liner disposed on the adhesive surface of the end body and covering the adhesive surface.

6. The fastening device of claim 5, wherein the adhesive surface is located on a first side of the adherable anchor and wherein the handle extends from the release liner to a second side of the adherable anchor opposite the first side.

7. The fastening device of claim 6, wherein the handle comprises a plurality of strips joined at a junction, each strip extending from a release liner disposed on the adhesive surface of the end body.

8. The fastening device of claim 7, wherein the release liner extends from a first edge to a second edge of the end body, the first edge disposed between the attachment portion and the second edge, and wherein each strip of the plurality of strips is connected to the release liner adjacent to the first edge and wherein the handle is configured to remove the release liner in response to pulling of the handle away from the adherable anchor, thereby exposing the adhesive surface of each arm.

9. The fastening device of claim 5, wherein each arm includes at least one of:
a cutout extending between the attachment portion and the end body; and
a plurality of struts connecting the attachment portion to the end body.

10. The fastening device of claim 5, wherein the attachment portion and the arms have a formed shape and are made of a flexible material configured to return to the formed shape when a force is removed.

11. The fastening device of claim 10, wherein the attachment portion is disposed in a first plane and the end body of each arm is disposed in a second plane, the second plane substantially parallel to the first plane and separated from the first plane by a distance, wherein the distance is less than a thickness of the wearable sensor such that the end bodies are disposed above a sensor face of the wearable sensor upon assembly with the wearable sensor.

12. The fastening device of claim 5, wherein the attachment portion comprises a hole configured to receive a mounting member projecting from a surface of the wearable sensor.

13. The fastening device of claim 5, wherein the end body comprises:
a first adhesive layer disposed in contact with the end body; and
a second adhesive layer disposed on the first adhesive layer, the second adhesive layer forming the adhesive surface.

14. The fastening device of claim 5, wherein the adherable anchor comprises an anti-rotation member configured to retain a position of the wearable sensor with respect to the adherable anchor, wherein the anti-rotation member comprises tabs that extend outward from the attachment portion.

15. The fastening device of claim 5, and further comprising a plurality of sinching members, each sinching member disposed on an arm and configured to apply pressure to one or more locations of the wearable sensor to tilt a sensor face of the wearable sensor toward a target area of interest.

16. The fastening device of claim 1, and further comprising an inflatable bladder disposed under the attachment portion and configured to apply pressure to one end of the wearable sensor to tilt a sensor face of the wearable sensor toward a target area of interest.

17. The fastening device of claim 1, and further comprising:
a rigid plate disposed on the attachment portion, the rigid plate having a threaded hole; and
a threaded fastener received in the threaded hole and configured to apply pressure to the wearable sensor to tilt a sensor face of the wearable sensor toward a target area of interest.

18. The fastening device of claim 1, and further comprising a wedge-shaped block disposed between the attachment portion and the wearable sensor and configured to tilt a sensor face of the wearable sensor toward a target area of interest.

19. A method of attaching a wearable sensor to a body of a patient, the wearable sensor comprising a sensor configured to measure characteristics inside the patient's body, the method comprising:
attaching a fastening device to the wearable sensor, the fastening device comprising:
an adherable anchor configured to affix the wearable sensor to skin of the body of the patient; and
a handle comprising a release liner attached to the adherable anchor;
positioning the wearable sensor on the body of the patient; and
pulling a handle of the fastening device to remove the release liner from an adhesive surface of the adherable anchor.

20. The method of claim 19, and further comprising tilting the sensor toward a target area of interest by one of:
inflating a bladder disposed between an attachment portion of the adherable anchor and a top surface of the wearable sensor to apply pressure to a portion of the top surface of the wearable sensor;
turning a threading a threaded fastener disposed through the adherable anchor and in contact with the top surface of the wearable sensor to apply pressure to a portion of the top surface of the wearable sensor;
tightening a cinching member of the adherable anchor to apply pressure to a portion of the top surface of the wearable sensor; and
providing a wedge-shaped block between the adherable anchor the top surface of the wearable sensor.
